(19) United States
(12) Patent Application Publication
(10) Pub. No.: US 2025/0376704 A1
(43) Pub. Date: Dec. 11, 2025

(54) BUTANOL PRODUCTION METHOD

(71) Applicant: NIPPON SHOKUBAI CO., LTD., Osaka (JP)

(72) Inventors: Masaharu MUKOYAMA, Tokyo (JP); Yusei NAGAMURA, Tokyo (JP)

(73) Assignee: NIPPON SHOKUBAI CO., LTD., Osaka (JP)

(21) Appl. No.: 18/875,309

(22) PCT Filed: Jun. 16, 2023
(86) PCT No.: PCT/JP2023/022392

§ 371 Date: Dec. 16, 2024

(30) Foreign Application Priority Data
Jun. 17, 2022 (JP) 2022-098493

(51) Int. Cl. C12P 7/16; B01D 3/00; B01D 3/36; B01D 61/36; B01D 71/24

(52) U.S. Cl. CPC C12P 7/16; B01D 3/002; B01D 3/36; B01D 61/362; B01D 71/24; B01D 2311/02; B01D 2311/06; B01D 2311/08

(57) ABSTRACT

Provided is a novel butanol production method. The butanol production method includes a fermentation step of subjecting a fermentation raw material to a fermentation treatment to prepare a fermented liquid containing butanol, and a separation step of subjecting the fermented liquid to PV membrane separation to prepare a separated liquid containing butanol. In the fermentation step, a microorganism of the species Clostridium saccharoperbutylacetonicum deficient in the function of at least an acetone-producing enzyme gene is used and/or a fermented liquid having an acetone concentration of 0.05 mass % or less is prepared.

### TECHNICAL FIELD

The present invention relates to a novel butanol production method and the like.

### BACKGROUND ART

Butanol fermentation is fermentation that utilizes bacteria to produce butanol primarily from carbohydrates.

Meanwhile, a pervaporation (PV) method is a membrane separation method in which a liquid is evaporated through a membrane, and a method for recovering a target product from a fermented liquid by such a PV method is also known (Patent Document 1 and the like).

### CITATION LIST

### Patent Document

Patent Document 1: JP 2010-161987 A

### SUMMARY OF INVENTION

### Technical Problem

An object of the present invention is to provide a novel butanol production method and the like.

### Solution to Problem

As described in Patent Document 1, the PV method is known as one method for recovering 1-butanol from a fermented liquid (culture liquid).

Under such circumstances, the present inventors have found that, when 35 butanol is recovered from a fermented liquid by the PV method, butanol can be efficiently recovered by, for example, using a specific microorganism or setting the composition of the fermented liquid or a liquid to be subjected to PV membrane separation to a specific one (adjusting the composition to a specific one), and have completed the present invention.

Accordingly, the present invention relates to the following inventions and the like.
[1]

A butanol production (recovery, separation) method including:
a fermentation step of subjecting a fermentation raw material to a fermentation treatment to prepare a fermented liquid containing butanol; and
a separation step of subjecting the fermented liquid (fermented liquid prepared through the fermentation step) to pervaporation (PV) membrane separation to prepare a separated liquid containing butanol, wherein
a microorganism of the species Clostridium saccharoperbutylacetonicum deficient in a function of at least an acetone-producing enzyme gene is used in the fermentation step.
[2]

A butanol production (recovery, separation) method including:
a fermentation step of subjecting a fermentation raw material to a fermentation treatment to prepare a fermented liquid containing butanol; and
a separation step of subjecting the fermented liquid (fermented liquid prepared through the fermentation step) to pervaporation (PV) membrane separation to prepare a separated liquid containing butanol, wherein
a fermented liquid having an acetone concentration of 0.05 mass % or less is prepared in the fermentation step (or a fermented liquid having an acetone concentration of 0.05 mass % or less is subjected to the PV membrane separation in the separation step).
[3]

The method according to [1] or [2], wherein, in the fermentation step, a microorganism of the species Clostridium saccharoperbutylacetonicum deficient in functions of at least an acetone-producing enzyme gene, a butyric acid-producing enzyme gene, and an acetic acid-producing enzyme gene is used.
[4]

The method according to any of [1] to [3], wherein, in the fermentation step, a fermented liquid containing (substantially) no acetone and having a butanol concentration of from 0.05 to 2 mass % is prepared.
[5]

The method according to any of [1] to [4], wherein, in the fermentation step, a fermented liquid containing (substantially) no acetone and having a butanol concentration of from 0.05 to 2 mass % and an ethanol concentration of 0.001 mass % or more is prepared.
[6]

The method according to any of [1] to [5], wherein, in the separation step, the separation (PV membrane separation) is performed at a pressure of from 0.1 to 50 kPa and a temperature (fermented liquid temperature, supply liquid temperature) of from 10 to 50° C.
[7]

The method according to any of [1] to [6], wherein, in the separation step, the separation (PV membrane separation) is performed so that a concentration factor of butanol is 15 times or more, and a ratio (X/Y) of a concentration factor X of butanol to a concentration factor Y of ethanol is 1.5 or more.
[8]

The method according to any of [1] to [7], wherein, in the separation step, a separated liquid is prepared, the separated liquid being phase-separated at least into a layer (e.g., an upper layer) 1 mainly containing butanol and a layer (e.g., a lower layer) 2 mainly containing water.
[9]

The method according to any of [1] to [8], wherein, in the separation step, a separated liquid having a butanol concentration of 6 mass % or more is prepared.
[10]

The method according to any of [1] to [9], wherein, in the separation step, a separated liquid containing (substantially) no acetone and having a butanol concentration of 6.5 mass % or more and a concentration of a non-aqueous component other than butanol (e.g., mainly ethanol) of 1 mass % or less is prepared.
[11]

The method according to any of [1] to [10], wherein a silicone rubber membrane is used as a separation membrane in the separation step.
[12]

The method according to any of [1] to [11], wherein the fermentation step and the separation step are carried out continuously.
[13]

The method according to any of [1] to [12], wherein
the fermentation step and the separation step are carried out continuously;
in the fermentation step, a microorganism of the species Clostridium saccharoperbutylacetonicum deficient in functions of at least an acetone-producing enzyme gene, a butyric acid-producing enzyme gene, and an acetic acid-producing enzyme gene is used to prepare a fermented liquid containing (substantially) no acetone and having a butanol concentration of from 0.1 to 1.5 mass % and an ethanol concentration of from 0.001 to 0.5 mass %; and
in the separation step, a silicone rubber membrane is used as a separation membrane, and at a pressure of from 0.3 to 10 Pa and a temperature of from 10 to 50° C., the separation (PV membrane separation) is performed so that a concentration factor of butanol is 20 times or more and a ratio (X/Y) of a concentration factor X of butanol to a concentration factor Y of ethanol is 1.8 or more, to thereby prepare
a separated liquid containing (substantially) no acetone and having a butanol concentration of 7 mass % or more and a concentration of a non-aqueous component (e.g., mainly ethanol) other than butanol of 0.5 mass % or less, the separated liquid being phase-separated at least into a layer (e.g., an upper layer) 1 mainly containing butanol and a layer (e.g., a lower layer) 2 mainly containing water.
[14]

The method according to any of [1] to [13], including a step of removing (reducing) carbon dioxide (dissolved carbon dioxide) from the fermented liquid to be subjected to the separation step (carbon dioxide removal (reduction) step) [wherein a fermented liquid from which carbon dioxide has been removed (reduced) (which has undergone the carbon dioxide removal (reduction) stop) is used as the fermented liquid to be subjected to the separation step].
[15]

The method according to any of [1] to [14], including a step (circulation step) of circulating (returning), to an appropriate stage or step [e.g., fermentation step (culture tank)] before the separation step, vapor (unrecovered vapor) which passes through a pervaporation membrane and is not liquefied in the separation step.
[16]

The method according to any of [1] to [15], further including a distillation step of distilling the separated liquid (separated liquid prepared in the separation step) (distillation step of distilling the separated liquid to separate and recover butanol).
[17]

The method according to any of [1] to [16], further including a distillation step of distilling the separated liquid, wherein
the separated liquid used in the distillation step is phase-separated at least into a layer (e.g., an upper layer) 1 mainly containing butanol and a layer (e.g., a lower layer) 2 mainly containing water, and the distillation step includes a distillation step 1 of distilling the layer 1 and a distillation step 2 of distilling the layer 2.
[18]

The method according to any of [1] to [17], wherein
the method does not include a step (distillation step or the like) of preliminarily separating a non-aqueous component other than butanol from the separated liquid;
the method further includes a distillation step of distilling the separated liquid; and
the separated liquid used in the distillation step is phase-separated at least into a layer (e.g., an upper layer) I mainly containing butanol and a layer (e.g., a lower layer) 2 mainly containing water, and the distillation step includes a distillation step 1 of distilling the layer 1 and a distillation step 2 of distilling the layer 2.
[19]

The method according to any of [1] to [18], further including a distillation step of distilling the separated liquid, wherein
in the distillation step, a partially removed (e.g., purged) separated liquid (or a separated liquid from which ethanol has been removed (reduced)) is distilled [further including a step of partially removing (e.g., purging) the separated liquid to be subjected to distillation (or a step of removing (reducing) ethanol)].
[20]

The method according to any of [1] to [19], wherein
the method does not include a step (a distillation step or the like) of preliminarily separating a non-aqueous component other than butanol from the separated liquid;
the method further includes a distillation step of distilling the separated liquid;
the separated liquid used in the distillation step is phase-separated at least into a layer (e.g., an upper layer) 1 mainly containing butanol and a layer (e.g., a lower layer) 2 mainly containing water, and the distillation step includes a distillation step 1 of distilling the layer 1 and a distillation step 2 of distilling the layer 2;
the method further includes a recovery (re-recovery) step of returning an azeotropic mixture 1 in the distillation step 1 and an azeotropic mixture 2 in the distillation step 2 to the separated liquid (supplying, returning them to recover (re-recover) butanol); and
in the distillation step, a separated liquid in which at least the layer 2 is partially removed (e.g., purged) (or a separated liquid in which ethanol is removed (reduced)) is used.

### Advantageous Effects of Invention

According to the present invention, it is possible to provide a novel butanol production method (recovery method).

In such a method, butanol can be efficiently recovered from a fermented liquid.

For example, when a specific microorganism is used in the fermentation, or when the composition of the fermented liquid or the liquid to be subjected to the PV membrane separation is set to a specific one (e.g., one having an extremely low acetone concentration), the separated liquid subjected to PV separation can be a separated liquid from which butanol is efficiently and easily recovered.

According to the investigation by the present inventors, when butanol is recovered (recovered with high purity) from the separated liquid after the PV separation, an additional process [for example, an additional distillation for preliminarily removing acetone or the like from the separated liquid because of failure to distill butanol therefrom, or an additional process associated with deterioration of the PV separation membrane (e.g., a process for separating a component contained in the separation membrane, which has been released or leaked out, or a process for replacing the separation membrane itself)] may be required depending on the liquid to be subjected to the PV membrane separation. However, according to the method of the present invention, such an additional process can be reduced (without performing the process in particular), and butanol can be efficiently recovered.

### DESCRIPTION OF EMBODIMENTS

The method of the present invention includes at least a fermentation step of preparing a fermented liquid containing butanol, and a separation step of subjecting the fermented liquid to pervaporation (PV) membrane separation to prepare a separated liquid containing butanol. Hereinafter, the present invention will be described, including these steps.

### Fermentation Step

In the fermentation step, a fermented liquid (culture liquid) containing butanol (1-butanol, isobutanol, or the like) is prepared. Such a fermented liquid is produced by subjecting a fermentation raw material to a fermentation treatment.

The fermentation treatment is usually performed using a microorganism (a microorganism capable of producing butanol).

Such a microorganism (a bacterium or the like) is not particularly limited as long as it is a microorganism that produces (makes) butanol. Examples of the microorganism include known microorganisms such as bacteria (microorganisms) belonging to the genus Clostridium and microorganisms (fermentation bacterial strains and the like) in which a butanol-metabolizing gene has been recombined.

The microorganism may be a genetically modified microorganism, for example, a microorganism (fermentation bacterial strain or the like) subjected to a mutation treatment (e.g., mutation treatment for improving the yield of butanol), or a microorganism (fermentation bacterial strain or the like) having increased resistance (e.g., high resistance to butanol). In particular, the microorganism may be a microorganism deficient in the function of at least an acetone-producing enzyme gene.

Such microorganisms may be those described in, for example, Green et al., Microbiology., 142:2079, 1996; Nair et al., J. Bacteriol., 176:871, 1994; Sillers et al., Biotechnol Bioeng., 102:38, 2009; Lehmann et al., Appl Microbiol Biotechnol., 94:743, 2012; Jang et al., mbio 2012., 23:00314, 2012; WO 2007/041269; U.S. Pat. No. 6,358,717; or JP 2014-207885 A.

Among these, microorganisms of the genus Clostridium, particularly microorganisms of the species Clostridium saccharoperbutylacetonicum (particularly genetically modified ones) may be suitably used.

Clostridium saccharoperbutylacetonicum (C. saccharoperbutylacetonicum) has an ability to produce butanol, and its strain is not particularly limited. Specific examples thereof include ATCC27021 strain and ATCC13564 strain.

In general, introduction of a plasmid into a microorganism of the genus Clostridium is difficult, but a plasmid can be relatively easily introduced into C. saccharoperbutylacetonicum. In C. acetobutylicum ATCC824 strain or the like, a plasmid cannot be introduced as it is because it is cleaved by DNA-endonuclease, and methylation treatment is required. However, in C. saccharoperbutylacetonicum, such a treatment is not required.

The microorganism (particularly, Clostridium saccharoperbutylacetonicum) may be one deficient in the function of at least an acetone-producing enzyme gene. By making the acetone-producing enzyme gene deficient, acetone is not produced as a by-product, and as described above, the butanol recovery process can be facilitated in combination with the PV separation. Further improvement in yield can be expected by combination with reducing power supply culture.

According to the investigation by the present inventors, the production of acetone (in addition, the production of acetic acid, butyric acid, or the like) can be a factor that impairs the recovery process of butanol in combination with PV membrane separation. However, such a microorganism efficiently and easily suppresses the production of acetone (in addition, acetic acid, butyric acid or the like) by genetic modification without impairing the efficient production of butanol, and can be suitably used in the fermentation step of the present invention.

The acetone-producing enzyme gene is a gene encoding an enzyme involved in a pathway for producing acetone from acetoacetyl-CoA. Examples of the acetone-producing enzyme gene include ctfA (gene encoding A subunit of CoA transferase), ctfB (gene encoding B subunit of CoA transferase), and adc (gene encoding acetoacetate decarbonylase). The CoA transferase is an enzyme that includes A subunit and B subunit and catalyzes a reaction of converting acetoacetyl-CoA into acetoacetate. The “ctfAB” refers to both the gene encoding A subunit and the gene encoding B subunit of CoA transferase. The acetoacetate decarbonylase is an enzyme that catalyzes a reaction of decarboxylating acetoacetate to produce acetone. Making the function of an acetone-producing enzyme gene deficient includes making deficient the function of a single gene among these genes, and making deficient the functions of a plurality of genes, and also includes making deficient one or a plurality of functions of a gene encoding an enzyme subunit.

As described above, the microorganism (particularly, Clostridium saccharoperbutylacetonicum) is preferably deficient in the function of at least an acetone-producing enzyme gene, but may be deficient in the function of a gene for an enzyme producing a substance other than acetone, simultaneously with acetone or independently of acetone.

For example, the microorganism (particularly, Clostridium saccharoperbutylacetonicum) may be deficient in the function of a butyric acid-producing gene. This makes the butanol recovery process easier in combination with the PV membrane separation.

The butyric acid-producing enzyme gene is a gene encoding an enzyme involved in a pathway for producing butyric acid from butyryl-CoA. Examples of the butyric acid-producing enzyme gene include ptb (gene encoding phosphotransbutyrylase) and buk (gene encoding butyrate kinase). The phosphotransbutyrylase is an enzyme that catalyzes a reaction of forming butyryl phosphate from butyryl-CoA. The butyrate kinase is an enzyme that catalyzes a reaction of converting butyryl phosphate to butyric acid. Making the function of a butyric acid-producing enzyme gene deficient includes making deficient the function of a single gene among these genes and making deficient the functions of a plurality of genes, and also includes making deficient one or a plurality of functions of a gene encoding an enzyme subunit.

Known examples in which the butyric acid production pathway is disrupted are those in C. acetobutylicum or the like. However, in such known examples, a large amount of acetic acid is produced due to disruption of the pathway for producing butyric acid, or an improvement in yield of butanol is not generally observed.

Meanwhile, in C. saccharoperbutylacetonicum, unexpectedly, when the butyric acid production pathway is disrupted, the phenomenon that acetic acid increases, as observed in C. acetobutylicum, is not observed, the bacterium grows well, and the butanol yield can be improved. As described above, the disruption of the butyric acid production pathway does not have a remarkable effect on butanol production in C. acetobutylicum, whereas the effects of reduction of by-products and improvement in butanol yield are obtained in C. saccharoperbutylacetonicum. Further, by disrupting the acetic acid production pathway of the strain in which the butyric acid production pathway is disrupted, the production of acetic acid and butyric acid can be greatly reduced, and the butanol yield can be greatly improved.

From this point of view, it is preferable to use Clostridium saccharoperbutylacetonicum as the microorganism even when the function of a butyric acid-producing gene is made deficient.

The microorganism (particularly, Clostridium saccharoperbutylacetonicum) may be deficient in the function of an acetic acid-producing gene. This makes it possible to further improve the butanol yield in the butanol fermentation.

The acetic acid-producing enzyme gene is a gene encoding an enzyme involved in a pathway for producing acetic acid from acetyl-CoA. Examples of the acetic acid-producing enzyme gene include pta (gene encoding phosphotransacetylase) and ack (gene encoding acetate kinase). The phosphotransacetylase is an enzyme that catalyzes a reaction of forming acetyl phosphate from acetyl-CoA. The acetate kinase is an enzyme that catalyzes a reaction of converting acetyl phosphate to acetic acid. Making the function of an acetic acid-producing enzyme gene deficient includes making deficient the function of a single gene among these genes and making deficient the functions of a plurality of genes, and also includes making deficient one or a plurality of functions of a gene encoding an enzyme subunit.

In addition, the microorganism (particularly, Clostridium saccharoperbutylacetonicum) may be deficient in the function of a lactic acid-producing gene. Even when lactic acid is produced, it can be converted into butanol again by metabolism.

The lactic acid-producing enzyme gene is a gene encoding an enzyme involved in a pathway for producing lactic acid from pyruvic acid. The lactic acid-producing enzyme gene includes lactate dehydrogenase. The lactate dehydrogenase is an enzyme that catalyzes interconversion of lactic acid and pyruvic acid. In this case, interconversion of NADH and NAD+ takes place simultaneously. The lactate dehydrogenase includes four different species. Two of them are cytochrome c dependent, and act on D-lactic acid (D-lactate dehydrogenase: EC 1.1.2.4) and L-lactic acid (L-lactate dehydrogenase: EC 1.1.2.3), respectively. The other two are NAD (P)-dependent enzymes, and act on D-lactic acid (D-lactate dehydrogenase: EC 1.1.1.28) and L-lactic acid (L-lactate dehydrogenase: EC 1.1.1.27), respectively. Specific examples of the lactate dehydrogenase include ldh1, ldh2, lldD and ldh3, and it is particularly preferable to make the function of ldh1 deficient. Making the function of a lactic acid-producing enzyme gene deficient includes making deficient the function of a single gene among these genes and making deficient the functions of a plurality of genes.

In particular, in the present invention, it is preferable to use a microorganism (particularly, a microorganism of the species Clostridium saccharoperbutylacetonicum) deficient in the function of at least one selected from an acetone-producing enzyme gene, a butyric acid-producing enzyme gene, and an acetic acid-producing enzyme gene. Especially, it is preferable to use a microorganism (in particular, a microorganism of the species Clostridium saccharoperbutylacetonicum) deficient in the function of at least an acetone-producing enzyme gene (preferably, at least an acetone-producing enzyme gene and a butyric acid-producing enzyme gene, and more preferably at least an acetone-producing enzyme gene, a butyric acid-producing enzyme gene, and an acetic acid-producing enzyme gene).

As used herein, the term “gene” includes DNA and RNA, and the term “DNA” includes single-stranded DNA and double-stranded DNA.

Making the function of an enzyme gene deficient includes modification (e.g., substitution, deletion, addition and/or insertion) or disruption of a part or the entirety of the enzyme gene to prevent an expression product of the gene from having the function as the enzyme, and to prevent an enzyme protein from being expressed. For example, it is possible to cause deletion, substitution, addition, or insertion in a part of the genomic DNA of an enzyme gene to make the function of the enzyme gene completely or substantially impaired or deficient. It also includes modification (e.g., substitution, deletion, addition, and/or insertion) or disruption of a part or the entirety of a promoter of an enzyme gene to prevent an enzyme protein from being expressed. Here, the state in which a gene is disrupted refers to a state in which a part or the entirety of the gene sequence is deleted, another DNA sequence is inserted into the gene sequence, or a partial sequence in the gene sequence is replaced with another sequence to make the function of the enzyme gene completely or substantially impaired.

A transformant of a microorganism (e.g., Clostridium saccharoperbutylacetonicum) deficient in the function of each enzyme gene is a knockout microorganism in which each enzyme gene on the genome is functionally impaired. Such a transformant can generally be prepared by using a known gene targeting method (e.g., Methods in Enzymology 225:803-890, 1993), for example, by homologous recombination. The method by homologous recombination can be carried out by inserting DNA of interest into a sequence homologous to a sequence on the genome, and introducing this DNA fragment into a cell to cause homologous recombination. When the DNA of interest is introduced into the genome, a strain in which homologous recombination has occurred can be easily selected by using a DNA fragment in which the DNA of interest is linked to a drug resistance gene. Alternatively, a DNA fragment in which a drug resistance gene is linked to a gene that becomes lethal under specific conditions can be inserted into the genome by homologous recombination, and then the drug resistance gene can be introduced in a form of replacing the gene that becomes lethal under specific conditions. In addition, a technique using a group II intron found in lactic acid bacteria (Guo et. al., Science 21; 289 (5478): 452-7 (2000)) or a genome processing method such as TALEN technology or CRISPR technology can also be used. In addition, techniques such as genome editing and point mutation are also available (WO2017/043656, JP 2020-22378 A, etc.).

The group II intron is an intron which forms a complex with a protein called LtrA of lactic acid bacteria and has the function of being inserted into a specific region in the genome. By appropriately altering a site referred to as targeting region of this intron, a DNA sequence can be inserted at a targeted location in the microbial genome. When the location where the DNA is inserted is inside a gene, the function of the gene is lost in most cases, and thus the technique can be used as a technique for gene disruption. In this case, when an appropriate drug resistance gene is inserted into the group II intron and a self-splicing DNA region called td intron is further inserted into the drug resistance gene, the drug resistance gene cannot be expressed in the form of a vector. However, after the group II intron containing the td intron is transcribed, the td intron is self-spliced out from the group II intron, and the group II intron without the td intron is inserted into genomic DNA, resulting in a functional drug resistance gene. The gene-disrupted strain obtained by this technique can be easily selected by using the drug resistance acquired by the drug resistance gene inserted into the genome as a marker.

A site called targeting sequence has been analyzed by Perutka et al. using Escherichia coli (Perutka et al., J. Mol. Biol. 13; 336 (2): 421-39 (2004)), and it has become possible to predict which site should be modified and how it should be inserted into a DNA sequence of interest. Based on this reference, for example, by programming an Excel macro and inputting the nucleotide sequence of the gene to be disrupted into this macro, the DNA insertion site into the gene of interest and the method for modifying the targeting sequence can be output.

In general, when a drug resistance gene is used for selection of a gene-disrupted strain, it is necessary to use another drug resistance gene for disruption of a plurality of genes. However, drug resistance can be eliminated by cutting out a drug resistance gene using the FLP-FRT method (Schweizer H P, J. Mol. Microbiol. Biotechnol. 5 (2): 67-77 (2003)), the Cre-loxP method (Hoess et al., Nucleic Acids Res. 11; 14 (5): 2287-300 (1986)), or the like. FLP and Cre have a function of recognizing short DNAs of about 25 bases called FRT and loxP, respectively, and cutting out a region sandwiched between FRTs or loxPs. That is, a drug-sensitive gene-disrupted strain can be obtained by carrying out gene disruption by placing an FRT sequence or a loxP sequence on a side part of a drug resistance gene in a vector for gene disruption, and then introducing another vector into which an FLP or Cre gene is cloned into the gene-disrupted strain which has become drug-resistant and allowing it to act thereon. Thereafter, gene disruption can be carried out again in the same manner.

Known sequences registered in the GenBank may be used as the sequences of the DNAs encoding the butyric acid-producing enzyme gene, the acetic acid-producing enzyme gene, the acetone-producing enzyme gene, and the lactic acid-producing enzyme gene. The nucleotide sequences of ctfA, ctfB, and adc of the C. saccharoperbutylacetonicum ATCC13564 strain are registered under Accession Number AY251646.

Genes functionally equivalent to the enzyme genes encoded by the above-described nucleotide sequences are also included in each enzyme gene. Examples of a gene functionally equivalent to an enzyme gene having a certain nucleotide sequence include a gene having a nucleotide sequence 70% or more, preferably 80% or more, more preferably 90% or more, still more preferably 95% or more, and most preferably 99% or more homologous (or identical) to the nucleotide sequence and encoding a protein having the same enzyme activity. For example, a gene functionally equivalent to ptb having SEQ ID NO: 1 includes a gene having a nucleotide sequence 70% or more, preferably 80% or more, more preferably 90% or more, still more preferably 95% or more, and most preferably 99% or more homologous to SEQ ID NO: 1 and encoding a protein having phosphotransbutyrylase activity. One skilled in the art can also use the reference numbers given in the GenBank for known genes to determine equivalent genes in other microorganisms.

A probe (e.g., from about 30 to 150 bases) can be prepared based on a known sequence, labeled with a radioactive or fluorescent label, and used to detect or isolate the genomic DNA of each enzyme gene. The genomic DNA is taken out from a microbial cell in accordance with a known method and then cleaved with a restriction enzyme. Thereafter, an open reading frame (ORF) of the enzyme gene of interest can be searched using the above probe by a hybridization method such as Southern hybridization or in situ hybridization. If necessary, a restriction enzyme map is prepared, an arbitrary target site for performing homologous recombination is determined, and a targeting vector is designed.

The vector for preparing a targeting vector by inserting a recombinant DNA is not particularly limited as long as it is a vector replicable in a microorganism (e.g., a microorganism of the genus Clostridium). A shuttle vector for E. coli and a microorganism of the genus Clostridium is convenient, and a shuttle vector such as pKNT19 derived from pIM13 (Journal of General Microbiology, 138, 1371-1378 (1992)) is particularly preferred.

A vector for obtaining a strain in which a gene is disrupted by transformation can be obtained by cloning a necessary sequence using microbial genomic DNA as a template, or by synthesizing the necessary sequence and appropriately linking them if necessary. A method for obtaining a desired gene or promoter from microbial genomic DNA by cloning is well known in the field of molecular biology. For example, when the sequence of the gene is known, a suitable genomic library can be made by restriction endonuclease digestion and screened using a probe complementary to the desired gene sequence. Once the sequence is isolated, the DNA can be amplified using a standard amplification method such as the polymerase chain reaction (PCR) (U.S. Pat. No. 4,683,202 B) to produce DNA in a quantity suitable for transformation. Methods for preparation of a genomic DNA library for use in cloning, hybridization, PCR, preparation of plasmid DNA, cleavage and linking of DNA, transformation, and the like are described in Sambrook, J., Fritsch, E. F., Maniatis, T., Molecular Cloning, Cold Spring Harbor Laboratory Press, 1.21 (1989). A DNA sequence can be directly synthesized, or a DNA sequence obtained by PCR or the like can be ligated by treatment with a restriction enzyme, or amplified by a PCR reaction using probes (primers) complementary to both ends of the DNA sequence to which a 15 bp-length homologous region of another DNA sequence is added, and subjected to an infusion reaction (U.S. Pat. No. 7,575,860 B), thereby being linked to obtain a DNA sequence having a longer chain.

Introduction of a targeting vector into a microorganism can be carried out by a known method. The method of introduction is not particularly limited, and examples thereof include a microcell method, a calcium phosphate method, a liposome method, a protoplast method, a DEAE-dextran method, and an electroporation method. The electroporation method is preferably used.

The fermentation treatment is usually performed using a microorganism (a microorganism capable of producing butanol), as described above.

Specifically, the fermentation treatment can be performed by fermenting a microorganism in the presence of a fermentation raw material (a butanol-producing raw material), and typically may be performed by culturing a microorganism in a medium (a medium containing a fermentation raw material, a medium capable of producing butanol) (culturing a microorganism and performing butanol fermentation). The fermentation (culture) can be carried out in an appropriate vessel (culture tank).

The fermentation raw materials, media, fermentation conditions (culture conditions), and the like used in such fermentation are not particularly limited, and may be those known in the field of butanol fermentation.

Examples of the fermentation raw material (fermentation raw material contained in the medium (culture medium)) include a carbon source, a nitrogen source, and an inorganic ion source. In general, the fermentation raw material (medium) may contain all of these.

As the carbon source, sugars such as monosaccharides, oligosaccharides, and polysaccharides are preferably used. Preferably, monosaccharides, in particular glucose is used. Glucose may be used in combination with other sugars such as lactose, galactose, fructose, or starch hydrolysate, alcohols such as sorbitol, or organic acids such as fumaric acid, citric acid, or succinic acid.

Examples of the nitrogen source include inorganic ammonium salts such as ammonium sulfate, ammonium chloride, and ammonium phosphate; organic nitrogen sources such as soybean hydrolysate, enzymatic decomposition products (tryptone and the like), amino acids, and peptide components; ammonia gas; and aqueous ammonia.

Examples of the inorganic ion source (inorganic ion) include a potassium ion (e.g., potassium phosphate), a magnesium ion (e.g., magnesium sulfate), an iron ion (e.g., iron sulfate), and a manganese ion.

In addition to these substances, the medium (fermentation raw material) may contain an organic micronutrient such as thiamin, p-aminobenzoic acid, vitamin B1, biotin, or another required substance, yeast extract or the like in an appropriate amount if necessary.

The medium is not particularly limited, and a commonly used medium such as a TYA medium (e.g., a medium described in Document A below or the like) or a P2 medium (e.g., a medium described in Document B below or the like) may be used. In addition, a medium obtained by substituting or mixing components used in such a medium (e.g., substituting medium components such as yeast extract and peptone with specific amino acids, nucleic acids, vitamins, and the like) may be used as the medium.

Document A (TYA medium):

Agric. Biol. Chem., 54 (2), 343-351, 1990

Document B (P2 medium):

Annous, B. A., and H. P. Blaschek. 1990. Regulation and localization of amylolytic enzymes in Clostridium acetobutylicum ATCC 824. Appl. Environ. Microbiol. 56:2559!2561.

In culture, the microorganism may be immobilized. For example, the microorganism may be immobilized on a carrier. The specific immobilizing means (carrier) is not particularly limited, and examples thereof include particles (e.g., organic particles and inorganic particles), natural polymers (e.g., cellulose, chitin, and chitosan), fibers (e.g., cellulose fibers, acrylic fibers, and nylon fibers), and porous carriers (e.g., sintered glass, pumice, and polyurethane foam).

The amount (proportion) of the fermentation raw material (fermentation raw material contained in the medium (culture medium)) can be appropriately determined depending on the type thereof or the like. For example, the amount (concentration) of the carbon source (e.g., sugar such as glucose) in the medium (fermented liquid) may be selected from a range of from about 1 to 1000 g/L (e.g., from 3 to 800 g/L, from 5 to 5700 g/L, from 10 to 500 g/L, from 50 to 800 g/L, from 100 to 700 g/L, or from 150 to 500 g/L), or may be from 1 to 300 g/L (e.g., from 1 to 250 g/L, from 1 to 200 g/L, or from 1 to 100 g/L), preferably from 3 to 100 g/L (for example, from 5 to 80 g/L, from 8 to 60 g/L, or from 10 to 40 g/L).

In particular, when the culture is performed continuously (when the culture is continuous culture) as described below, the amount (concentration) of the carbon source (e.g., sugar such as glucose) in the medium (medium to be continuously supplied) may be relatively high, and may be, for example, from 50 to 800 g/L, from 100 to 700 g/L, or from 150 to 500 g/L.

The transformant may be cultured under conditions in which the reducing power is increased. Under such conditions, it is easy to produce butanol with high efficiency while suppressing the production of by-products such as acetone, ethanol, acetic acid, butyric acid, and lactic acid, and the absolute production amount of butanol is also easy to increase. The culture under conditions in which the reducing power is increased means that the enzyme reaction occurring during culture is carried out under conditions in which the reducing power is increased. The reducing power can be increased by, for example, adding NADH, introducing hydrogen, increasing the hydrogen partial pressure in the culture tank, or the like.

In the fermentation (culture, medium), the pH is preferably 4.6 or more, 4.7 or more, 4.8 or more, 4.9 or more, 5 or more, or 5.5 or more, and may preferably be 8 or less, 7.5 or less, 7.0 or less, 6.9 or less, 6.8 or less, 6.7 or less, 6.6 or less, or 6.5 or less. By carrying out the culture while adjusting the pH in this manner, the production amount of butanol relative to by-products can be further increased.

For the pH adjustment, an inorganic or organic acidic or alkaline substance such as calcium carbonate, ammonia, sodium hydroxide, potassium hydroxide, or potassium phosphate can be used. The pH adjustment also includes a case where the medium is maintained at a pH of interest without adding an alkaline substance or the like as described above. For example, when ammonium sulfate is used as the nitrogen source, if it is replaced with ammonium acetate having a high buffering capacity, the decrease in pH may be suppressed and growth may be improved.

Other fermentation (culture) conditions are not particularly limited, and conditions commonly used in the art can be employed. For example, when batch culture is performed, the fermentation (culture) time is usually from 5 to 100 hours, and preferably from 12 to 48 hours. In the case of continuous culture or fed-batch culture, the fermentation (culture) time is usually 200 hours or more, preferably 500 hours or more, and more preferably 1000 hours or more. The fermentation (culture) temperature may be usually adjusted to from 20 to 55° C., preferably from 25 to 40° C., or the like.

In particular, from the viewpoint of efficient production (recovery) of butanol, the fermentation (culture) may be performed continuously (in a continuous manner) together with the separation step described below (associated with the separation step).

Examples of the method for performing the fermentation continuously include a method of returning (circulating) a residual liquid after the PV separation (a liquid which has not been subjected to the PV separation (which has not passed through the membrane)) to the fermentation step (culture tank) again while supplying the fermented liquid to the separation step described below.

Vapor which has passed (permeated) through the PV separation membrane and has not been liquefied [vapor (unrecovered vapor) which has not been liquefied (could not be recovered) as a PV separated liquid] may be discarded, or may be returned (circulated) to an appropriate stage or step before the PV separation [e.g., the fermentation step (culture tank)] from the viewpoint of butanol recovery efficiency or the like. By circulating the vapor in this manner, butanol can be recovered more efficiently, and this tendency is particularly remarkable, for example, when the condensation efficiency is low.

An object to be circulated (residual liquid, unrecovered vapor) may be at least a part thereof, and a part or the entirety of an object to be returned may be circulated.

In the fermentation (culture) [e.g., in the case of continuous fermentation (continuous culture)], each component (constituent component of the medium) may be appropriately supplemented.

In the fermentation step, a fermented liquid (culture liquid) is produced.

The fermented liquid contains butanol. The butanol concentration of the fermented liquid (or the butanol concentration after the culture) may be, for example, 0.01 mass % or more, preferably 0.05 mass % or more, and more preferably 0.1 mass % or more (e.g., 0.12 mass % or more).

The upper limit of the butanol concentration of the fermented liquid may be, for example, 2 mass % or less, 1.8 mass % or less, 1.5 mass % or less, 1.2 mass % or less, 1 mass % or less, 0.9 mass % or less, 0.8 mass % or less, 0.7 mass % or less, or 0.6 mass % or less.

In particular, the butanol concentration of the fermented liquid is preferably not too high from the viewpoint of survival of the microorganism and the like. Meanwhile, it is also preferably not too low from the viewpoint of production of butanol. In particular, when the fermentation step (and further the separation step) is performed continuously, such a preferable tendency is remarkable.

From such a viewpoint, the butanol concentration of the fermented liquid may be, for example, 0.05 mass % or more (e.g., 0.07 mass % or more, 0.08 mass % or more, 0.1 mass % or more, 0.12 mass % or more, or 0.13 mass % or more) and 2 mass % or less (e.g., 1.8 mass % or less, 1.5 mass % or less, 1.2 mass % or less, 1 mass % or less, 0.9 mass % or less, 0.8 mass % or less, 0.7 mass % or less, or 0.6 mass % or less).

Preferably, the fermented liquid contains no acetone (contains substantially no acetone). Even if it contains acetone, the amount thereof may be a very trace amount. For example, the acetone concentration of the fermented liquid may be 0.05 mass % or less [e.g., from 0 mass % (or the detection limit) to 0.03 mass %], preferably 0.01 mass % or less, and more preferably 0.005 mass % or less.

Such a fermented liquid containing no acetone or a very trace amount of acetone if any may be prepared by any method, and the above-described microorganism (e.g., Clostridium saccharoperbutylacetonicum deficient in the function of at least an acetone-producing enzyme gene) is not necessarily used. However, by using the above-described microorganism, a fermented liquid having the above-described acetone concentration (further, in addition to the butanol concentration, the below-described ethanol concentration, the concentrations of other components, and the like) is easily and efficiently produced.

The fermented liquid desirably contains no ethanol, but usually a trace amount of ethanol is contained (by-produced) in many cases. The ethanol concentration of such a fermented liquid may be, for example, 0.001 mass % or more (e.g., from 0.001 to 0.5 mass %), preferably 0.002 mass % or more (e.g., from 0.002 to 0.2 mass %), and more preferably about 0.003 mass % or more (e.g., from 0.003 to 0.1 mass %, 0.005 mass % or more), or may be 0.01 mass % or more (e.g., 0.02 mass % or more) or the like.

During fermentation, carbon dioxide is generated, and a part thereof can be dissolved in the culture liquid. When the concentration of carbon dioxide (dissolved carbon dioxide) in such a fermented liquid (fermented liquid to be subjected to the separation step) is set to a low concentration, the separation step (and thus a series of steps) described below can be performed more efficiently [e.g., butanol can be separated (condensed) with high efficiency without performing the separation step at an extremely low temperature].

Specifically, the concentration of carbon dioxide (dissolved carbon dioxide) in the fermented liquid [or the fermented liquid to be subjected to the separation step, or the fermented liquid in which carbon dioxide (dissolved carbon dioxide) is reduced] may be, for example, 0.3 mass % or less, preferably 0.2 mass % or less, and more preferably 0.15 mass % or less.

The method for adjusting (reducing, removing) such dissolved carbon dioxide (carbon dioxide removal step) is not particularly limited as long as it is a method that can be performed on the fermented liquid to be subjected to the separation step (a method that can be performed at a timing before the fermented liquid is subjected to the separation step). Examples of such a method include (1) a method of removing generated carbon dioxide gas, (2) a method of diffusing (volatilizing) carbon dioxide dissolved in the fermented liquid [e.g., (2A) a method of stirring a culture tank (culture liquid) (e.g., a method of culturing under the same degree of stirring as aerobic culture in a culture tank), (2B) a method of flashing from a culture tank to an intermediate tank and gasifying (releasing) dissolved carbon dioxide, (2C) an ultrasonic treatment method, (2D) a decompression method in an intermediate tank, and (2E) a method of stirring the fermented liquid in an intermediate tank], and (3) a combination of these methods.

The fermented liquid preferably does not contain (substantially does not contain) other components (volatile components) such as butyric acid, acetic acid, and lactic acid [components (volatile components, organic components) other than water, butanol, acetone, ethanol, and carbon dioxide (dissolved carbon dioxide)]. Even if it contains such components, the amount thereof may be a trace amount.

Specifically, the concentration of other components [volatile components other than water, butanol, acetone, ethanol, and carbon dioxide (dissolved carbon dioxide) (volatile organic components other than butanol, acetone, and ethanol)] in the fermented liquid may be, for example, 0.5 mass % or less, preferably 0.2 mass % or less, and more preferably 0.1 mass % or less.

The butyric acid concentration of the fermented liquid may be, for example, 0.1 mass % or less, preferably 0.07 mass % or less, and more preferably 0.05 mass % or less.

The acetic acid concentration of the fermented liquid may be, for example, 0.1 mass % or less, preferably 0.07 mass % or less, and more preferably 0.05 mass % or less.

The lactic acid concentration of the fermented liquid may be, for example, 0.2 mass % or less, preferably 0.1 mass % or less, and more preferably 0.05 mass % or less.

In the fermented liquid, the mass ratio of butanol to ethanol [butanol/ethanol (mass ratio)] may be, for example, 15 or more (e.g., from 15 to 100), preferably 20 or more (e.g., from 20 to 60), and more preferably 30 or more (e.g., from 30 to 50).

In the fermented liquid, the mass ratio of butanol to other components [volatile components other than water, butanol, acetone, ethanol, and carbon dioxide (volatile organic components other than butanol, acetone, and ethanol)] [butanol/other components (mass ratio)] may be, for example, 8 or more (e.g., from 8 to 100), preferably 10 or more (e.g., from 10 to 60), and more preferably 20 or more (e.g., from 20 to 40).

The mass ratio of butanol to carbon dioxide (dissolved carbon dioxide) [butanol/carbon dioxide (mass ratio)] in the fermented liquid [or the fermented liquid to be subjected to the separation step, or the fermented liquid in which carbon dioxide (dissolved carbon dioxide) is reduced] may be, for example, 20 or more (e.g., from 20 to 100), preferably 30 or more (e.g., from 30 to 80), and more preferably 40 or more (e.g., from 40 to 60).

In the fermented liquid, the mass ratio of ethanol to other components [volatile components other than water, butanol, acetone, ethanol, and carbon dioxide (dissolved carbon dioxide) (volatile organic components other than butanol, acetone, and ethanol)] [ethanol/other components (mass ratio)] may be, for example, 0.1 or more (e.g., from 0.1 to 2), preferably 0.15 or more (e.g., from 0.15 to 1), and more preferably 0.2 or more (e.g., from 0.2 to 0.8).

In the fermented liquid, the mass ratio of butanol to the total amount of acetone, ethanol, and other components [volatile components other than water, butanol, acetone, ethanol, and carbon dioxide (dissolved carbon dioxide) (volatile organic components other than butanol, acetone, and ethanol)] [butanol/(acetone, ethanol, and other components) (mass ratio)] may be, for example, 1 or more (e.g., from 1 to 100), preferably 2 or more (e.g., from 2 to 20), and more preferably 5 or more (e.g., from 5 to 10).

By preparing the above-described fermented liquid [e.g., the fermented liquid which contains no acetone or an extremely small amount of acetone if any (further, a fermented liquid in which the concentrations of butanol and other components are also adjusted)], butanol can be efficiently recovered in combination with the separation step described below. [For example, an additional step of separating and removing acetone and the like is likely to be unnecessary; a separated liquid which is two phase-separated is easily obtained by the separation step; the fermentation step is easily performed stably for a long period of time (particularly, continuously together with the separation step); the separation step is easily performed for a long period of time (particularly, continuously together with the fermentation step) even without using a special separation membrane; and/or the separation step is easily performed for a long period of time while suppressing deterioration of the separation membrane (particularly, continuously together with the fermentation step).

The concentration (proportion) or the like as described above may be satisfied in a part of the fermentation step, may be an average during the fermentation step, or may be satisfied during the entire fermentation step [satisfied (maintained) from the start to the end of the fermentation step].

For example, when the fermentation step is performed continuously, the fermented liquid may be supplied to the separation step (further, the liquid which has not been separated may be circulated) so that the concentration or the like as described above is achieved (the concentration is maintained). By adjusting the concentration in this manner, a series of steps (production, separation, recovery, and the like of butanol) can be performed more efficiently.

The concentration may be adjusted by addition (supply) of a component or a medium used in the fermentation step, discharge of a medium or a culture liquid, adjustment of the amount of supply to the separation step, circulation (recirculation) from other steps, or the like, or may be adjusted using an appropriate concentration adjusting means.

For example, in continuous culture, when a medium containing a fermentation raw material (e.g., sugar at a high concentration) is fed, the concentration of the fermentation raw material (e.g., sugar) in the culture tank may be adjusted (e.g., may be made not too high) by feeding a medium corresponding to the amount of the fermentation raw material (e.g., sugar) consumed in accordance with the consumption rate of the fermentation raw material (e.g., sugar) in the culture tank.

In such a case, the concentration of the fermentation raw material (e.g., sugar) in the culture tank can be made substantially zero by supplying the medium while monitoring the concentration of the fermentation raw material (e.g., sugar) in the culture tank using a feed controller combined with a sensor (e.g., a glucose sensor) or the like as necessary.

In the case of continuous culture, the balance between the amount of components such as butanol and water discharged (removed) from the culture tank and the amount of the medium to be supplied (added) may be adjusted.

In general, the amount of the continuously supplied medium may be larger than the amount of components such as butanol and water separated in the separation step (PV membrane).

In such a case, the culture liquid exceeding the volume of the culture tank needs to be discharged from the culture tank, and butanol contained in the discharged culture liquid may be separated and recovered by a method such as distillation and then discharged as a waste culture liquid as described below.

However, from the viewpoint of waste reduction, a higher concentration of the fermentation raw material (e.g., sugar) in the medium continuously supplied to the culture tank is preferred because the amount of the waste culture liquid is reduced. However, if the concentration of the fermentation raw material (e.g., sugar) in the medium is too high, the amount of waste culture liquid discharged is too small, resulting in a large accumulation of waste products in the medium, which may reduce the efficiency of butanol fermentation. Therefore, the culture liquid (e.g., culture liquid in such an amount that accumulation of waste products can be prevented) may be discharged as long as the fermentation is not inhibited.

When the culture liquid is discharged from the culture tank, a part or the entirety of the microorganism (bacterial cells) contained in the culture liquid can be separated and returned to the culture tank. For the separation, a method such as filtration or centrifugation can be employed. As described above, when the microorganism is immobilized and used or a cohesive microorganism (bacterial strain or the like) is used, the bacterial cells can be efficiently separated by a simple method such as filtration or centrifugation. Such operations of separating the microorganism and returning it to the culture tank are preferably carried out under anaerobic conditions.

A part of the culture liquid may be discharged without being subjected to the separation step. Since such a culture liquid contains butanol which is not separated in the separation step (PV membrane), the butanol may also be separated and recovered.

The method for separating and recovering butanol is not particularly limited, and for example, distillation using a mash column or the like is preferred. However, when a sugar remains in the culture liquid, the Maillard reaction, in which a sugar reacts with an amino acid and a protein, occurs due to heating during distillation, and the culture liquid becomes dark brown in color, and the load of waste medium treatment becomes very large. In order to prevent such a problem, it is desirable to reduce the concentration of the sugar contained in the waste medium as low as possible, and more preferably to the detection limit or less.

For this purpose, it is effective to use a method in which the medium is supplied to the culture tank with a feed controller while monitoring the sugar concentration in the culture tank so that the sugar concentration in the culture tank becomes substantially zero. A culture liquid having a sugar concentration of substantially zero can be introduced into an apparatus for distilling and recovering butanol such as a mash column, to thereby separate and recover butanol, and bacteria contained in the culture liquid can be killed by heating of the distillation column.

When a small amount of sugar remains in the culture tank, the culture liquid discharged from the culture tank is introduced into an aging tank, and allowed to stay therein and cultured (aging culture) under the same conditions as those in the culture tank for a predetermined time (e.g., from 1 to 24 hours) to completely consume the remaining sugar, and then butanol can be separated and recovered in a mash column or the like. When a relatively large amount (e.g., about from 10 to 30 g/L) of sugar remains in the culture liquid, it is desirable that aging culture is performed for a predetermined staying time (e.g., from 1 to 24 hours) by switching between two aging tanks, and then butanol is separated and recovered in a mash column or the like.

### Separation Step

In the separation step, the fermented liquid is subjected to pervaporation (PV) membrane separation to produce a separated liquid containing butanol.

In the PV membrane separation, the separation membrane (PV separation membrane, membrane material) is not particularly limited, and any of silicone (silicone rubber), zeolite, and the like can be used.

In the present invention, the fermented liquid as described above is subjected to the PV separation, and thus butanol can be efficiently separated even without using a special membrane (e.g., a membrane impregnated with a component for selective permeation of a desired component).

In addition, according to the fermented liquid as described above, the separation membrane seems to be less deteriorated, and the separation step can be stably performed for a long period of time.

For example, although a separation membrane made of silicone (silicone rubber membrane) is originally excellent in durability in many cases, the separation membrane may be easily deteriorated or may interfere with stable separation due to swelling or the like when a fermented liquid containing acetone is separated with the membrane. However, according to the above-described fermented liquid, the separation step can be easily continued stably without impairing the durability, even if a separation membrane made of silicone is used.

The shape of the PV separation membrane is not particularly limited. In addition to a flat membrane, a membrane in the form of a hollow fiber is used, and a bundled membrane is suitably used for increasing the surface area used for separation.

The PV separation membrane module (module including a PV separation membrane) is not particularly limited. For example, a module having a structure in which a PV separation membrane (the hollow fiber bundle or the like) is housed in a cylindrical case and both ends of the case are connected to a pipe connector, or a module in which hollow fibers are bundled in a sheet shape is used.

The thickness of the PV separation membrane may be, for example, from 10 to 1000 μm, preferably from 20 to 500 μm, and more preferably from 30 to 400 pm (e.g., from 40 μm to 200 μm). In the present invention, even when a separation membrane having such a thickness is used (and even when the temperature of the fermented liquid to be subjected to the PV separation is not increased), the PV membrane separation can be efficiently performed with high separation efficiency.

The fermentation step and the separation step can be carried out separately, but are preferably carried out continuously (in association with each other, simultaneously). When the fermentation step and the separation step are carried out simultaneously, continuous fermentation becomes possible, and thus a culture tank cleaning operation or the like required in batch production is not required. Therefore, only the number of such operations can be reduced, whereby a significant reduction in production cost can be achieved. In addition, since a large amount of water can be usually removed in the separation step, the amount of overflowing fermented liquid is reduced even when a nutrient source is continuously added and supplied to a fermentation tank, and thus it is possible to easily confine the microorganism in the fermented liquid, which is advantageous because the loss of microorganism and the amount of waste water are reduced.

As a mode of combination of PV separation and culture, for example, in the case of a cylindrical module or the like, the module is disposed outside the fermentation tank, and the fermented liquid is introduced from the fermentation tank to the module by using a liquid feeding pump. Continuous production of butanol (continuous fermentation step) becomes possible by returning the liquid from which butanol has been separated to the fermentation tank. The liquid from which butanol has been separated may be temporarily stored in a storage tank and used again as the fermentation raw material. In the case of a sheet-form module, the module can be directly incorporated into the fermentation tank, and thus equipment such as a liquid feeding pump is not required, and the design of the entire production apparatus can be made compact.

As the fermented liquid (supply liquid) to be subjected to the PV separation, the fermented liquid produced in the fermentation step can be supplied as it is, but the fermented liquid may be a liquid after completion of the fermentation. Alternatively, it is also possible to use a liquid obtained by removing the microorganism (bacterial cell) from the liquid in which fermentation is proceeding by a method such as centrifugation, membrane separation, or immobilization. The method for removing the microorganism is not particularly limited, and an appropriate (e.g., appropriately simple) method can be selected depending on the mode of the microorganism (e.g., whether the microorganism is immobilized on a carrier).

Conditions (operation conditions) of the separation step are not particularly limited, and may be selected depending on the composition of the fermented liquid (supply liquid) to be subjected to the separation, the composition of the separated liquid obtained after the separation, or the like.

For example, the temperature of the fermented liquid to be subjected to the separation step (fermented liquid at the time of the PV separation) may be selected depending on, for example, the separation efficiency, or whether or not the supply liquid contains a microorganism. Specifically, when, for example, the supply liquid contains microorganisms (further (a part of) the supply liquid after separation is returned to the fermentation step), the temperature of the fermented liquid (supply liquid) may be selected from a range of from about 5 to 100° C. (e.g., from 10 to 80° C. or from 20 to 70° C.), or may be from 10 to 50° C. (e.g., from 15 to 45° C., from 20 to 40° C., from 25 to 35° C., or from 30 to 40° C.). In the present invention, the PV separation can be efficiently performed even at such a temperature, and thus the fermentation step and the separation step can be efficiently performed continuously.

In particular, when the fermented liquid (culture liquid) containing a microorganism is subjected to PV separation, the temperature of the fermented liquid (temperature during PV separation) may be a temperature (e.g., in the range of from 25 to 40° C.) at which the fermentation activity of the microorganism can be efficiently maintained.

Meanwhile, when the fermented liquid (culture liquid) from which the microorganism has been removed (in which the microorganism is not contained) is subjected to the PV separation, the temperature of the fermented liquid (temperature at the time of the PV separation) may be, for example, from 10 to 100° C., preferably from 30 to 80° C., and more preferably from 50 to 70° C. from the viewpoint of the permeation rate or the like.

In the separation step, the pressure (pressure on a permeation and evaporation side of the membrane) may be usually low (vacuum, reduced pressure) for promoting evaporation of butanol. The same effect can also be obtained by supplying a gas (e.g., N₂, H₂, CO₂ gas, a gas generated by fermentation, or a mixed gas thereof) as a carrier (carrier gas) to the permeation and evaporation side of the membrane. Further, the carrier gas supply and vacuum may be applied simultaneously.

When the pressure is low, the pressure at the time of separation (degree of vacuum or reduced pressure) can also be selected depending on the separation efficiency and the composition of the separated liquid as described above, and a suitable pressure can also be selected in combination with the above-described temperature and the like. A specific pressure can be selected from a range of, for example, 50 kPa or less, and may be from 0.1 to 50 kPa, preferably from 0.3 to 10 kPa, and more preferably from 0.5 to 5 kPa.

As described above, the fermented liquid produced in the fermentation step contains butanol and a trace amount of ethanol, and even if acetone or the like is contained, the amount thereof is a very trace amount in many cases. For such a fermented liquid, when the pressure is in such a range (particularly in combination with the temperature), butanol can be more efficiently recovered (e.g., the concentration factor of butanol can be selectively increased).

The liquid (vapor) that has passed through the PV separation membrane is obtained as a separated liquid (liquefied separated liquid). Such liquefaction may be caused by allowing to cool or by cooling (cooling treatment). For the cooling (treatment), a cooler (cooling trap) or the like can be appropriately used. The cooling temperature is not particularly limited, and may be, for example, 10° C. or lower, 5° C. or lower, 0° C. or lower, or lower than 0° C. (−2° C. or lower, or −5° C. or lower).

As described above, the separated liquid [the liquid having passed through the PV separation membrane (the liquid obtained by liquefying vapor)] is produced. In the fermented liquid (supply liquid), the liquid that has not been separated (liquid other than the separated liquid) may be recycled by, for example, circulation to the fermentation step (fermentation tank, culture tank) as described above.

The mode (composition, concentration, or the like) of the separated liquid depends on the fermented liquid (supply liquid) and the PV separation conditions, and is, for example, as follows.

First, the separated liquid contains butanol. In general, the separated liquid may contain water in addition to butanol.

The butanol concentration of the separated liquid can be selected from a range of about 0.1 mass % or more (e.g., 0.5 mass % or more), and may be, for example, 1 mass % or more (e.g., 2 mass % or more), preferably 3 mass % or more (e.g., 4 mass % or more), and more preferably 5 mass % or more (e.g., 6 mass % or more, 6.5 mass % or more, 7 mass % or more, 7.5 mass % or more, or 8 mass % or more).

The upper limit of the butanol concentration of the separated liquid may be, for example, 50 mass % or less, 40 mass % or less, 30 mass % or less, 25 mass % or less, 20 mass % or less, 18 mass % or less, or 15 mass % or less.

Although depending on the components other than butanol in the separated liquid, the concentrations thereof, and the like, two phase separation described below is likely to occur at a predetermined butanol concentration (e.g., a relatively high concentration such as 6 mass % or more). Therefore, the butanol concentration (further, the concentrations of other components described below and the concentration factor described below in relation to the fermented liquid) may be adjusted so that phase separation such as two phase separation occurs.

The concentration factor X of butanol [the ratio (X2/X1) of a butanol concentration (mass) X2 in the separated liquid to a butanol concentration (mass) X1 in the fermented liquid (supply liquid)] may be adjusted depending on the PV separation conditions, whether to cause two phase separation, or the like, and may be selected from a range of, for example, about 5 times or more, and may be 10 times or more, preferably 15 times or more, and more preferably 20 times or more (e.g., 22 times or more, or 25 times or more). With such a concentration factor, efficient recovery of butanol is easily realized in the process from the fermentation step to the separation step.

The separated liquid preferably contains no acetone (contains substantially no acetone), and, even if it contains acetone, the amount thereof may be a very trace amount. For example, the acetone concentration of the separated liquid may be 0.2 mass % or less, preferably 0.1 mass % or less, and more preferably 0.05 mass % or less.

As described above, the fermented liquid usually contains no acetone or a very trace amount of acetone if any, and acetone can be separated to some extent also in the PV separation. Reflecting this, the separated liquid usually contains no acetone or a very trace amount of acetone if any.

The separated liquid desirably does not contain ethanol, and, even if it contains ethanol, the amount thereof may be a very trace amount. The ethanol concentration in such a separated liquid may be, for example, 0.01 mass % or more (e.g., 0.01 to 0.5 mass %), preferably 0.05 mass % or more (e.g., 0.05 to 0.4 mass %), and more preferably about 0.07 mass % or more (e.g., 0.1 to 0.3 mass %), and may be 0.5 mass % or less (e.g., 0.4 mass % or less, 0.3 mass % or less, 0.28 mass % or less, 0.25 mass % or less, 0.2 mass % or less, 0.18 mass % or less, or 0.15 mass % or less).

The concentration factor Y of ethanol [the ratio (Y2/Y1) of an ethanol concentration (mass) Y2 in the separated liquid to an ethanol concentration (mass) Y1 in the fermented liquid (supply liquid)] may be adjusted depending on the PV separation conditions or the like, and may be, for example, 1.2 times or more (e.g., 1.5 times or more, 2 times or more, 3 times or more, or 5 times or more), and may be 50 times or less (e.g., 40 times or less, 35 times or less, 30 times or less, 25 times or less, 20 times or less, or 15 times or less).

The ratio (X/Y) of the concentration factor X of butanol to the concentration factor Y of ethanol may preferably be more than 1, or may be 1.1 or more (e.g., 1.2 or more), 1.5 or more (e.g., 1.6 or more, 1.8 or more, 2 or more, 2.2 or more, 2.4 or more, or 2.5 or more). The upper limit of X/Y is not particularly limited, and may be, for example, 20 or less, 18 or less, 15 or less, 12 or less, 10 or less, 9 or less, 8 or less, 7 or less, or 6 or less.

That is, the ethanol concentration of the separated liquid reflects the fermented liquid to some extent in many cases, and the degree of the reflection can be efficiently reduced, as compared with butanol, through the PV separation (and thus butanol can be efficiently separated).

The separated liquid preferably does not contain (substantially does not contain) other components (volatile components) such as butyric acid, acetic acid, and lactic acid [components (volatile components, organic components) other than water, butanol, acetone, and ethanol] or carbon dioxide (dissolved carbon dioxide), and, even if it contains such components, the amount thereof may be a trace amount.

Specifically, the concentrations of other components [volatile components other than water, butanol, acetone, ethanol, and carbon dioxide (dissolved carbon dioxide) (volatile organic components other than butanol, acetone, and ethanol)] and carbon dioxide (dissolved carbon dioxide) in the separated liquid may be, for example, 1 mass % or less, preferably 0.5 mass % or less, and more preferably 0.1 mass % or less.

The butyric acid concentration of the separated liquid may be, for example, 0.5 mass % or less, preferably 0.1 mass % or less, and more preferably 0.01 mass % or less.

The acetic acid concentration of the separated liquid may be, for example, 1 mass % or less, preferably 0.1 mass % or less, and more preferably 0.01 mass % or less.

The lactic acid concentration of the separated liquid may be, for example, 1 mass % or less, preferably 0.1 mass % or less, and more preferably 0.01 mass % or less.

The carbon dioxide (dissolved carbon dioxide) concentration of the separated liquid may be, for example, 0.1 mass % or less, preferably 0.05 mass % or less, and more preferably 0.01 mass % or less.

The ratio (X/Z) of the concentration factor X of butanol to the concentration factor Z of the other components may preferably be more than 1, or may be 1.1 or more (e.g., 1.2 or more), 1.5 or more (e.g., 1.6 or more, 1.8 or more, 2 or more, 2.2 or more, 2.4 or more, or 2.5 or more). The upper limit of X/Y is not particularly limited, and may be, for example, 20 or less, 18 or less, 15 or less, 12 or less, 10 or less, 9 or less, 8 or less, 7 or less, or 6 or less.

In the separated liquid, the concentration of non-aqueous components other than butanol (e.g., the total amount of ethanol and non-aqueous components other than ethanol) may be, for example, 3 mass % or less (e.g., 2.5 mass % or less), preferably 2 mass % or less (e.g., 1.5 mass % or less), and more preferably 1 mass % or less (e.g., 0.8 mass % or less, 0.5 mass % or less, 0.4 mass % or less, 0.3 mass % or less, 0.2 mass % or less, 0.15 mass % or less, 0.12 mass % or less, or 0.1 mass % or less).

The concentration of non-aqueous components other than butanol in the separated liquid is usually lower than the concentration of butanol.

In the separated liquid, the mass ratio of butanol to ethanol [butanol/ethanol (mass ratio)] may be, for example, 15 or more (e.g., from 15 to 200), preferably 20 or more (e.g., from 20 to 150), and more preferably 30 or more (e.g., from 30 to 100).

In the separated liquid, the mass ratio of butanol to other components [volatile components other than water, butanol, acetone, ethanol, and carbon dioxide (dissolved carbon dioxide) (volatile organic components other than butanol, acetone, and ethanol)] and carbon dioxide (dissolved carbon dioxide) [butanol/other components and carbon dioxide (dissolved carbon dioxide) (mass ratio)] may be, for example, 90 or more (e.g., from 90 to 600), preferably 100 or more (e.g., from 100 to 500), and more preferably 120 or more (e.g., from 120 to 400).

In the separated liquid, the mass ratio of ethanol to other components [volatile components other than water, butanol, acetone, ethanol, and carbon dioxide (dissolved carbon dioxide) (volatile organic components other than butanol, acetone, and ethanol)] and carbon dioxide (dissolved carbon dioxide) [ethanol/other components and carbon dioxide (dissolved carbon dioxide) (mass ratio)] may be, for example, 0.5 or more (e.g., from 0.5 to 10), preferably 1 or more (e.g., from 1 to 6), and more preferably 1.5 or more (e.g., from 1.5 to 5).

In the separated liquid, the mass ratio of butanol to the total amount of acetone, ethanol, carbon dioxide (dissolved carbon dioxide), and other components [volatile components other than water, butanol, acetone, ethanol, and carbon dioxide (dissolved carbon dioxide) (volatile organic components other than butanol, acetone, and ethanol)] [butanol/(acetone, ethanol, and other components) (mass ratio)] may be, for example, 5 or more (e.g., from 5 to 30), preferably 7 or more (e.g., from 7 to 25), and more preferably 10 or more (e.g., from 10 to 20).

The separated liquid may be phase-separated. When the separated liquid is phase-separated, butanol can be more easily recovered from the separated liquid.

A typical example of such a separated liquid is a separated liquid obtained by at least phase separation (particularly, two phase separation) into a layer (e.g., an upper layer) 1 mainly containing butanol and a layer (e.g., a lower layer) 2 mainly containing water.

In the layer mainly containing butanol, the concentration of butanol may be, for example, 70 mass % or more (e.g., from 70 to 90 mass %), preferably 75 mass % or more (e.g., from 75 to 85 mass %), and more preferably about 78 mass % or more (e.g., from 78 to 82 mass %).

In the layer mainly containing water, the concentration of butanol may be, for example, 9 mass % or less (e.g., from 6 to 9 mass %), preferably 8.5 mass % or less (e.g., from 6.5 to 8.5 mass %), and more preferably about 8 mass % or less (e.g., from 7 to 8 mass %).

### Other Steps

The method of the present invention may include steps other than the fermentation step and the separation step.

For example, the method of the present invention may further include a step of separating and recovering butanol from the separated liquid (recovery step, separation and recovery step). In such a step, specific examples of the recovery (purification) method include known methods such as distillation and extraction.

A typical example of the recovery step is a distillation step of distilling the separated liquid (separated liquid produced in the separation step) (distillation step of distilling the separated liquid to separate and recover butanol). In the distillation, equipment, distillation conditions, and the like are not particularly limited, and a known method can be used.

Prior to the distillation step, the separated liquid may be subjected to a step of preliminarily separating non-aqueous components (e.g., other components) other than butanol [non-butanol (e.g., other component) separation step]. However, in the present invention, the separated liquid can usually be subjected to the distillation step without undergoing such a non-butanol separation step.

The distillation step can be selected depending on the form of the separated liquid, and, for example, the distillation of the separated liquid which is phase-separated as described above may be performed for each separated layer to recover butanol.

As an example of such a distillation step, when the separated liquid is phase-separated at least into a layer (an upper layer) 1 mainly containing butanol and a layer (a lower layer) 2 mainly containing water, the distillation step may include a distillation step 1 of distilling the upper layer 1 and a distillation step 2 of distilling the lower layer 2.

When such layer-by-layer distillation is carried out, butanol is recovered mainly from the upper layer 1 (e.g., recovered from the bottom of the distillation column or as a bottom liquid). Meanwhile, in the distillation step 2, a mixture (azeotropic mixture) containing butanol is taken out and used for further recovery of butanol in many cases.

Typically, the method of the present invention may include a recovery (re-recovery) step of returning (supplying, returning and recovering (re-recovering) butanol) an azeotropic mixture 1 in the distillation step 1 and an azeotropic mixture 2 in the distillation step 2 to the separated liquid.

As described above, the fermentation step and the separation step are preferably performed continuously. However, the other steps as described above may also be performed continuously.

The separated liquid (separated liquid to be subjected to the distillation step) may be used in its entirety, or may be used (subjected to the distillation step) after a part of the separated liquid is removed (extracted and discarded). Examples of the method (treatment) for removing a part of the separated liquid include purging (purge treatment). These treatments may be performed alone, or a combination of two or more thereof may be performed.

In particular, at least purging (treatment) may be performed.

The method and conditions (e.g., the type of gas, and the purge ratio) of the partial removal (purging or the like) are not particularly limited, and can be appropriately selected depending on the component (e.g., ethanol) to be removed, the amount thereof, and the like.

For example, the purge ratio may be selected from a range of about 0.1 mass % or more (e.g., 0.5 mass % or more), and may be, for example, 1 mass % or more, preferably 2 mass % or more, and more preferably 2.5 mass % or more. The upper limit of the purge ratio may be, for example, 5 mass % or less, 4 mass % or less, or 3.5 mass % or less.

The purge ratio is a ratio of the liquid to be removed with respect to the entire liquid to be purged (e.g., a layer mainly containing water in the separated liquid which is phase-separated when the layer is subjected to a purge treatment).

The partial removal (purging or the like) may be performed on at least a part of the separated liquid. For example, the partial removal may be performed on the entire separated liquid, or a part of the separated liquid [e.g., one or more layers (e.g., a layer mainly containing water) of the separated liquid which is phase-separated].

In particular, it is preferable to remove (purge or the like) at least a part of the layer mainly containing water in the separated liquid which is phase-separated.

When the separated liquid is distilled, ethanol or the like may be contained as an azeotropic composition (e.g., in addition to the entire separated liquid, ethanol comes out from the top as an azeotropic composition containing butanol, ethanol, and water in the distillation of the layer mainly containing water, or ethanol comes out from the top as an azeotropic composition containing butanol, ethanol, and water in the distillation of the layer mainly containing butanol).

Therefore, when the process (butanol recovery) is repeated while, for example, the azeotropic composition is utilized, ethanol or the like may be accumulated in the separated liquid to be subjected to distillation [the entire separated liquid, at least one layer (particularly, the layer mainly containing water) of the separated liquid which is phase-separated], but such accumulation can be efficiently prevented or suppressed by performing the above-described treatment.

The liquid from which butanol has been recovered can be reused as appropriate depending on its components and the like. For example, as described above, the mixture containing butanol can be returned to the separated liquid again (and then subjected to the distillation step), and the separated water can also be used in the fermentation step or the like.

In the present invention, butanol can be produced (recovered, purified) as described above.

Butanol can be used as a raw material (butanol source) for various compounds [e.g., butyl ester (e.g., butyl acrylate), butyl ether (e.g., ethylene glycol monobutyl ether)] in addition to the case where butanol is used as it is. Therefore, butanol (butanol produced by the method of the present invention) may be a raw material (butanol source) for various compounds or the like.

Such butanol (butanol produced by the method of the present invention) is produced from a fermentation raw material (biobutanol), and is also useful in terms of environment (e.g., in terms of suppression of carbon dioxide emission). In particular, biobutanol produced by fermentation using a component (e.g., sugar) obtained by decomposition of biomass that is a renewable resource as a raw material (or various compounds using the biobutanol as a raw material) is very useful as contributing to suppression of carbon dioxide emission.

The present invention is not limited to the above-described embodiments, and various modifications can be made, and embodiments obtained by appropriately combining technical means disclosed in different embodiments are also included in the present invention.

### EXAMPLES

The present invention will be described in more detail below by way of Examples, but is not limited to these Examples.

### Examples

In the same manner as in Example 1 of JP 2014-207885 A, microorganisms of the species Clostridium saccharoperbutylacetonicum (Clostridium saccharoperbutylacetonicum ATCC27021 ΔptaΔptb1ΔctfB strain) in which the functions of an acetone-producing enzyme gene, a butyric acid-producing enzyme gene and an acetic acid-producing enzyme gene were deficient were obtained and cryopreserved.

A culture apparatus in which a PV separation membrane (module) indicated below was connected to a 2 L jar fermenter was provided.

Membrane used: silicone membrane module M40-6000 available from Nagayanagi Co., Ltd., hollow fiber internal diameter: 0.17 mm, membrane thickness: 0.04 mm, membrane area: 0.55m² (intermediate value between internal and external diameters)

Then, each step was performed as follows.

First, 1 mL of a frozen stock solution of microorganisms (Clostridium saccharoperbutylacetonicum ATCC27021 ΔptaΔptb1ΔctfB strain) was thawed, inoculated into 9 ml of a fermenting medium having a composition (concentration) shown in the following table, and statically cultured for 24 hr at 30° C. Then, the whole amount was inoculated into 100 ml of a medium having the same composition and cultured at 30° C. for 16 hours. Up to this time point, the operation was performed in an anaerobic glove box.

TABLE 1

| | Per L | |
|---|---|---|
| Tryptone | 6 | g |
| Yeast extract | 2 | g |
| Glucose | 55 | g |
| (NH₄)₂SO₄ | 2.58 | g |
| MgSO₄•7H₂O | 0.3 | g |
| KH₂PO₄ | 0.5 | g |
| FeSO₄•7H₂O | 10 | mg |
| ADEKA NOL | 0.2 | mL |
| 2N-NaOH | Amount to achieve pH 6.5 | |
| Tap water | 1 | L |

Thereafter, the whole amount of 100 ml of a culture was inoculated into the previously prepared and sterilized 2 L volume jar fermenter, and cultivation was started at 30° C. and 100 rpm. From 15 minutes before the inoculation to 30 minutes after the inoculation, N2 gas was passed through the medium at 1 vvm, and an anaerobic state was maintained. A fed-batch medium shown in the following table (4 times the concentration of the above-described medium) was supplied from 16 hr of culture at a flow rate set to 0.5 g/min (the flow rate between 50 and 144 hr was temporarily set at 0.7 g/min).

TABLE 2

| | Per L | |
|---|---|---|
| Tryptone | 24 | g |
| Yeast extract | 8 | g |
| Glucose | 220 | g |
| (NH₄)₂SO₄ | 10.32 | g |
| MgSO₄•7H₂O | 1.2 | g |
| KH₂PO₄ | 02 | g |
| FeSO₄•7H₂O | 40 | mg |
| ADEKA NOL | 0.8 | mL |
| 2N-NaOH | Amount to achieve pH 6.5 | |
| Tap water | 1 | L |

The fermented liquid was subjected to PV membrane separation under the following conditions to produce a separated liquid.
Circulated liquid amount: 100 ml/min
Pressure (reduced pressure): 15 torr
Circulated liquid (supply liquid) temperature: 30° C.
Cooling temperature: −5° C.
Supply liquid: produced fermented liquid (2 L jar fermenter culture liquid)

The PV membrane separation was started (from the fermented liquid) 19 hours after the start of the culture.

During the continuous process described above, the separated liquid was recovered stepwise (cumulatively) and observed, and the separated liquid recovered at any stage was generally separated into two phases.

In the continuous process, the fermented liquid and the separated liquid after the elapse of a predetermined period of time were picked up and recovered, and the composition of each liquid was measured. The concentration factor and the like were also calculated from the measured values.

The composition (concentration) of each liquid was measured by a gas chromatograph GC-2010 available from Shimadzu Corporation (DB-WAX. column available from Agilent Technologies).

The results are shown in the table below.

In the following table, “wt %” represents mass (weight) %; “BuOH” represents 1-butanol; “EtOH” represents ethanol; “Concentration factor” represents a concentration after the PV membrane separation (concentration in the separated liquid)/concentration in the fermented liquid; “Concentration factor ratio” represents the concentration factor of BuOH/the concentration factor of EtOH; “Incremental BuOH” represents the amount of BuOH increased from the amount of BuOH in the separated liquid measured immediately before; “Cumulative BuOH” represents the cumulative (integrated) amount of BuOH produced from the start of fermentation (PV membrane separation); and “Separated liquid” represents the total amount of the separated liquid produced by the PV membrane separation (PV separated liquid).

Although not shown in the following table, each of the acetone concentration and the butyric acid concentration of the fermented liquid was 0 mass % (detection limit), and therefore, each of these concentrations in the PV separated liquid was naturally 0 mass % (detection limit). Similarly, the acetic acid concentration of the fermented liquid was 0.2 mass % at most, and the acetic acid concentration of the PV separated liquid was 0.1 mass % (detection limit). In addition, the lactic acid concentration of the fermented liquid was 0.1 mass % at most, and the lactic acid concentration of the PV separated liquid was 0 mass % (detection limit).

Further, the concentration of carbon dioxide (dissolved carbon dioxide) in the fermented liquid was about 0.5 mass % at most, and the concentration of carbon dioxide (dissolved carbon dioxide) in the fermented liquid to be subjected to separation was 0.15 mass % or less.

The adjustment (reduction) of dissolved carbon dioxide was performed by stirring the culture tank (stirring rate: from 200 to 300 rpm).

TABLE 3

| | Fermented | | PV membrane separation | | | | | | | |
|---|---|---|---|---|---|---|---|---|---|---|
| | liquid (in | | BuOH | | EtOH | | Concen- | | | |
| Elapsed | medium) | | Concen- | Concen- | Concen- | Concen- | tration | Recovery amount | | |
| time | BuOH | EtOH | tration | tration | tration | tration | factor | Incremental | Cumulative | Separated |
| (hours) | (wt %) | (wt %) | (wt %) | factor | (wt %) | factor | ratio | BuOH (g) | BuOH (g) | liquid (g) |
| 51 | 0.16 | 0.0043 | 7.19 | 44.1 | 0.078 | 18.3 | 2.4 | 39.76 | 39.76 | 168.0 |
| 67 | 0.26 | 0.0058 | 10.20 | 38.7 | 0.106 | 18.2 | 2.1 | 31.07 | 70.83 | 304.7 |
| 75 | 0.54 | 0.0163 | 13.84 | 25.7 | 0.135 | 8.3 | 3.1 | 15.74 | 86.57 | 113.7 |
| 91 | 0.39 | 0.0092 | 12.74 | 33.0 | 0.128 | 13.9 | 2.4 | 31.37 | 117.94 | 246.2 |
| 97.5 | 0.38 | 0.0119 | 12.45 | 32.4 | 0.127 | 10.6 | 3.0 | 12.57 | 130.52 | 101.0 |
| 115 | 0.22 | 0.0057 | 8.29 | 37.8 | 0.089 | 15.7 | 2.4 | 28.89 | 159.41 | 348.5 |
| 163 | 0.23 | 0.0078 | 9.14 | 39.2 | 0.089 | 11.4 | 3.4 | 59.30 | 218.71 | 293.8 |
| 172.5 | 0.21 | 0.0099 | 11.19 | 53.1 | 0.113 | 11.4 | 4.7 | 20.14 | 238.84 | 179.9 |
| 187 | 0.29 | 0.0079 | 13.10 | 45.5 | 0.133 | 16.8 | 2.7 | 35.00 | 273.84 | 267.2 |
| 195 | 0.25 | 0.0127 | 12.31 | 49.5 | 0.123 | 9.7 | 5.1 | 17.63 | 291.47 | 143.2 |
| 211 | 0.23 | 0.0053 | 9.58 | 42.0 | 0.097 | 18.2 | 2.3 | 25.01 | 316.48 | 261.2 |
| 219.5 | 0.21 | 0.0070 | 9.40 | 44.6 | 0.092 | 13.1 | 3.4 | 12.23 | 328.70 | 130.0 |
| 259 | 0.32 | 0.0139 | 12.55 | 39.6 | 0.130 | 9.4 | 4.2 | 44.69 | 373.39 | 198.9 |
| 266 | 0.30 | 0.0130 | 13.30 | 44.1 | 0.131 | 10.1 | 4.4 | 11.25 | 384.64 | 84.6 |
| 283 | 0.28 | 0.0148 | 12.44 | 44.3 | 0.124 | 8.4 | 5.3 | 24.50 | 409.14 | 196.9 |
| 315.5 | 0.31 | 0.0119 | 13.98 | 45.5 | 0.137 | 11.6 | 3.9 | 34.61 | 443.75 | 247.7 |
| 338 | 0.21 | 0.0068 | 8.84 | 42.7 | 0.089 | 13.1 | 3.3 | 15.54 | 459.29 | 175.7 |
| 385 | 0.66 | 0.0218 | 24.90 | 38.0 | 0.245 | 11.2 | 3.4 | 35.44 | 494.73 | 106.52 |
| 405.5 | 0.59 | 0.0323 | 25.08 | 42.7 | 0.268 | 8.3 | 5.1 | 19.15 | 513.88 | 76.36 |

As is apparent from the results shown in the above table, the process of the above Example made it possible to produce butanol continuously and efficiently (to recover a liquid containing about 514 g of butanol in 405.5 hours of culture).

For example, acetone or the like was not contained in the fermented liquid (culture liquid), and ethanol contained in a trace amount was also significantly reduced by the PV membrane separation. In addition, the fermentation (culture) and butanol production were continuously performed, and the fermentation was not delayed.

There was a correlation between the butanol concentration of the fermented liquid (culture liquid) and the butanol concentration of the PV separated liquid; i.e., the higher the concentration in the fermented liquid, the higher the concentration in the PV separated liquid.

As described above, the PV separated liquid did not contain acetone or the like and was two phase-separated, and thus was a separated liquid capable of easily and efficiently recovering butanol by distillation (distillation of each phase) without requiring a distillation process for separation of acetone or the like.

Further, after the completion of the process, the membrane (silicone membrane module) used in the PV membrane separation was observed, but no deterioration or the like was particularly determined. These results indicated that the above process can be realized over a long period of time.

### INDUSTRIAL APPLICABILITY

According to the present invention, a novel butanol production method and the like can be provided.

1. A butanol production method comprising:
a fermentation step of subjecting a fermentation raw material to a fermentation treatment to prepare a fermented liquid containing butanol; and
a separation step of subjecting the fermented liquid to pervaporation membrane separation to prepare a separated liquid containing butanol, wherein
a microorganism of the species Clostridium saccharoperbutylacetonicum deficient in a function of at least an acetone-producing enzyme gene is used in the fermentation step.

2. A butanol production method comprising:
a fermentation step of subjecting a fermentation raw material to a fermentation treatment to prepare a fermented liquid containing butanol; and
a separation step of subjecting the fermented liquid to pervaporation membrane separation to prepare a separated liquid containing butanol, wherein
a fermented liquid having an acetone concentration of 0.05 mass % or less is prepared in the fermentation step.

3. The method according to claim 1, wherein, in the fermentation step, a microorganism of the species Clostridium saccharoperbutylacetonicum deficient in functions of at least an acetone-producing enzyme gene, a butyric acid-producing enzyme gene, and an acetic acid-producing enzyme gene is used.

4. The method according to claim 1, wherein, in the fermentation step, a fermented liquid containing substantially no acetone and having a butanol concentration of from 0.05 to 2 mass % is prepared.

5. The method according to claim 1, wherein, in the fermentation step, a fermented liquid containing substantially no acetone and having a butanol concentration of from 0.05 to 2 mass % and an ethanol concentration of 0.001 mass % or more is prepared.

6. The method according to claim 1, wherein, in the separation step, the separation is performed at a pressure of from 0.1 to 50 kPa and a temperature of from 10 to 50° C.

7. The method according to claim 1, wherein, in the separation step, the separation is performed so that a concentration factor of butanol is 15 times or more, and a ratio (X/Y) of a concentration factor X of butanol to a concentration factor Y of ethanol is 1.5 or more.

8. The method according to claim 1, wherein, in the separation step, a separated liquid is prepared, the separated liquid being phase-separated at least into a layer 1 mainly containing butanol and a layer 2 mainly containing water.

9. The method according to claim 1, wherein, in the separation step, a separated liquid having a butanol concentration of 6 mass % or more is prepared.

10. The method according to claim 1, wherein, in the separation step, a separated liquid containing substantially no acetone and having a butanol concentration of 6.5 mass % or more and a concentration of a non-aqueous component other than butanol of 1 mass % or less is prepared.

11. The method according to claim 1, wherein a silicone rubber membrane is used as a separation membrane in the separation step.

12. The method according to claim 1, wherein the fermentation step and the separation step are carried out continuously.

13. The method according to claim 1, wherein the fermentation step and the separation step are carried out continuously;
in the fermentation step, a microorganism of the species Clostridium saccharoperbutylacetonicum deficient in functions of at least an acetone-producing enzyme gene, a butyric acid-producing enzyme gene, and an acetic acid-producing enzyme gene is used to prepare a fermented liquid containing substantially no acetone and having a butanol concentration of from 0.1 to 1.5 mass % and an ethanol concentration of from 0.001 to 0.5 mass %; and
in the separation step, a silicone rubber membrane is used as a separation membrane, and at a pressure of from 0.3 to 10 Pa and a temperature of from 10 to 50° C., the separation is performed so that a concentration factor of butanol is 20 times or more and a ratio (X/Y) of a concentration factor X of butanol to a concentration factor Y of ethanol is 1.8 or more, to thereby prepare a separated liquid containing substantially no acetone and having a butanol concentration of 7 mass % or more and a concentration of a non-aqueous component other than butanol of 0.5 mass % or less, the separated liquid being phase-separated at least into a layer 1 mainly containing butanol and a layer 2 mainly containing water.

14. The method according to claim 1, comprising a step of removing carbon dioxide from the fermented liquid to be subjected to the separation step.

15. The method according to claim 1, comprising a step of circulating, to the fermentation step, vapor which passes through a pervaporation membrane and is not liquefied in the separation step.

16. The method according to claim 1, further comprising a distillation step of distilling the separated liquid.

17. The method according to claim 1, further comprising a distillation step of distilling the separated liquid, wherein
the separated liquid used in the distillation step is phase-separated at least into a layer 1 mainly containing butanol and a layer 2 mainly containing water, and the distillation step includes a distillation step 1 of distilling the layer 1 and a distillation step 2 of distilling the layer 2.

18. The method according to claim 1, wherein
the method does not comprise a step of preliminarily separating a non-aqueous component other than butanol from the separated liquid;
the method further comprises a distillation step of distilling the separated liquid; and
the separated liquid used in the distillation step is phase-separated at least into a layer 1 mainly containing butanol and a layer 2 mainly containing water, and the distillation step includes a distillation step 1 of distilling the layer 1 and a distillation step 2 of distilling the layer 2.

19. The method according to claim 1, further comprising a distillation step of distilling the separated liquid, wherein
a purged separated liquid is distilled in the distillation step.

20. The method according to claim 1, wherein
the method does not comprise a step of preliminarily separating a non-aqueous component other than butanol from the separated liquid;
the method further comprises a distillation step of distilling the separated liquid;
the separated liquid used in the distillation step is phase-separated at least into a layer 1 mainly containing butanol and a layer 2 mainly containing water, and the distillation step includes a distillation step 1 of distilling the layer 1 and a distillation step 2 of distilling the layer 2;
the method further comprises a recovery step of returning an azeotropic mixture 1 in the distillation step 1 and an azeotropic mixture 2 in the distillation step 2 to the separated liquid; and
in the distillation step, a separated liquid in which at least the layer 2 is purged is used.
